(19) United States
(12) Patent Application Publication
(10) Pub. No.: US 2010/0273734 A1
(43) Pub. Date: Oct. 28, 2010

(54) Derivatives of Hyaluronic Acids

(75) Inventor: Feng Xu, Davis, CA (US)

Correspondence Address: NOVOZYMES, INC., 1445 DREW AVE, DAVIS, CA, 95618, US

(73) Assignee: Novozymes Biopolymer A/S, Bagsvaerd (DK)

(21) Appl. No.: 11/680,057

(22) Filed: Feb. 28, 2007

Related U.S. Application Data
(60) Provisional application No. 60/777,819, filed on Feb. 28, 2006.

(51) Int. Cl. A61K 31/728; C07H 5/06; A61K 8/73; A61P 17/02; A61P 17/14; A61P 19/10; A61P 27/02; A61P 35/00; A61Q 19/00; A61Q 90/00

(52) U.S. Cl. 514/54; 536/55.2

(57) ABSTRACT

The present invention relates to methods for preparing a derivative of a hyaluronic acid, comprising: (a) mixing a liquid solution comprising the hyaluronic acid and a diamine, a polyamine, or a combination thereof, at a pH suitable to form an imine; (b) reducing the imine to an amine with a reductant at a pH suitable to produce the derivative of the hyaluronic acid; and (c) recovering the derivative of the hyaluronic acid. The present invention also relates to isolated derivatives of a hyaluronic acid, comprising the hyaluronic acid and a diamine, a polyamine, or a combination thereof.

### CROSS-REFERENCE TO RELATED APPLICATION

This application claims the benefit of U.S. Provisional Application No. 60/777,819, filed Feb. 28, 2006, which application is incorporated herein by reference.

### BACKGROUND OF THE INVENTION

1. Field of the Invention

The present invention relates to derivatives of a hyaluronic acid and methods for preparing the derivatives of the hyaluronic acid.

2. Description of the Related Art

Hyaluronic acid (HA) is a natural and linear carbohydrate polymer belonging to the class of non-sulfated glycosaminoglycans. It is composed of beta-1,3-N-acetyl glucosamine and beta-1,4-glucuronic acid repeating disaccharide units with molecular weights up to 10 MDa. Hyaluronic acid is present in hyaline cartilage, synovial joint fluid, and skin tissue, both dermis and epidermis, and can be extracted from natural tissues including connective tissue of vertebrates, human umbilical cord, and rooster combs.

Numerous roles of hyaluronic acid have been identified in the human body (see, Laurent T. C. and Fraser J. R. E., 1992, FASEB J. 6: 2397-2404; and Toole B. P., 1991, “Proteoglycans and hyaluronan in morphogenesis and differentiation,” In: Cell Biology of the Extracellular Matrix, pp. 305-341, Hay E. D., ed., Plenum, N.Y.). It plays an important role as a mechanical support for cells of many tissues, such as skin, tendons, muscles and cartilage. Hyaluronic acid is involved in key biological processes, such as the moistening of tissues and lubrication. It is also suspected of being involved in numerous physiological functions, such as adhesion, development, cell motility, cancer, angiogenesis, and wound healing. Due to the unique physical and biological properties of hyaluronic acid (including viscoelasticity, biocompatibility, and biodegradability), hyaluronic acid is employed in a wide range of current and developing applications within cosmetics, opthamology, rheumatology, drug and gene delivery, wound healing, and tissue engineering.

The water-binding capacity and viscoelastic property of hyaluronic acid are important in its use as a biomaterial. These properties are controlled by the concentration and molecular weight of hyaluronic acid.

High molecular weight hyaluronic acid has been traditionally extracted from rooster combs and bovine vitreous humor, but it often forms a complex with proteoglycans, making its purification difficult (O'Regan et al., 1994, International Journal of Biological Macromolecules 16: 283-286). Alternatively, hyaluronic acid can be produced by bacterial fermentation processes. While Streptococcus strains are known to produce high molecular hyaluronic acid, the strains are often virulent and pathogenic, making purification difficult and expensive. Recombinant methods involving Bacillus host cells can also be used to produce hyaluronic acid (U.S. Pat. No. 6,951,743, WO 03/0175902), but hyaluronic acid so produced reportedly has an average molecular weight in the range of 1 to 2 MDa or less.

The use of hyaluronic acid in several of the above applications is limited by the availability of hyaluronic acid having a suitable molecular weight to generate desirable viscoelastic, mechanical, stability, and/or matrix/carrier properties. For example, ophthalmic or osteoarthritic applications can require a hyaluronic acid of 4 MDa or higher (Wobig et al., 1999, Clin Ther. 21: 1549-1562; Armstrong et al., 1997, Applied and Environmental Microbiology 63: 2759-2764; Goa and Benfield, 1994, Drugs 47: 536-566; Swann and Kuo, 1991, Hyaluronic acid, p. 286-305, In D. Byrom (ed.), Biomaterials-novel materials from biological sources, Stockton Press, New York, N.Y.; U.S. Pat. No. 4,784,990), and cosmetic applications can require a hyaluronic acid of 2-4 MDa (Swann and Kuo, 1991, supra; U.S. Pat. No. 4,784,990). Consequently, there is a need in the art for methods to make derivatives of hyaluronic acid with higher average molecular weights.

It is an object of the present invention to provide new methods for making derivatives of a hyaluronic acid of varying average molecular weights.

### SUMMARY OF THE INVENTION

The present invention relates to methods for preparing a derivative of a hyaluronic acid, comprising:
(a) mixing a liquid solution comprising the hyaluronic acid and a diamine, a polyamine, or a combination thereof, at a pH suitable to form an imine;
(b) reducing the imine to an amine with a reductant at a pH suitable to produce the derivative of the hyaluronic acid; and
(c) recovering the derivative of the hyaluronic acid.

The present invention also relates to isolated derivatives of a hyaluronic acid, comprising the hyaluronic acid and a diamine, a polyamine, or a combination thereof.

The present invention also relates to compositions comprising such a hyaluronic acid derivative and an inactive component(s), an active component(s), or a combination of an inactive component(s) and an active component(s).

The present invention also relates to cosmetic and sanitary articles comprising such a hyaluronic acid derivative or a composition thereof.

The present invention also relates to a medicament capsule, comprising such a hyaluronic acid derivative or a composition thereof.

### BRIEF DESCRIPTION OF THE FIGURES

FIG. 1 shows the structural formula of the repeating disaccharide unit of N-acetylglucosamine (GlcNAc) and glucuronic acid (GlcUA) in hyaluronic acid.

FIG. 2 shows the reaction of a hyaluronic acid with a diamine or a polyamine to produce an imine.

FIG. 3 shows reduction of an imine with borohydride as the reductant to produce an amine.

FIG. 4 shows a derivative of a diamine and a hyaluronic acid wherein R′ is either H or NHCOCH₃, R″ is either CO₂H or CH₂OH, and R is the rest of the structure of a diamine.

### DETAILED DESCRIPTION OF HE INVENTION

The present invention relates to methods for preparing a derivative of a hyaluronic acid, comprising: (a) mixing a liquid solution comprising the hyaluronic acid and a diamine, a polyamine, or a combination thereof, at a pH suitable to form an imine, (b) reducing the imine to an amine with a reductant at a pH suitable to produce the derivative of the hyaluronic acid; and (c) recovering the derivative of the hyaluronic acid.

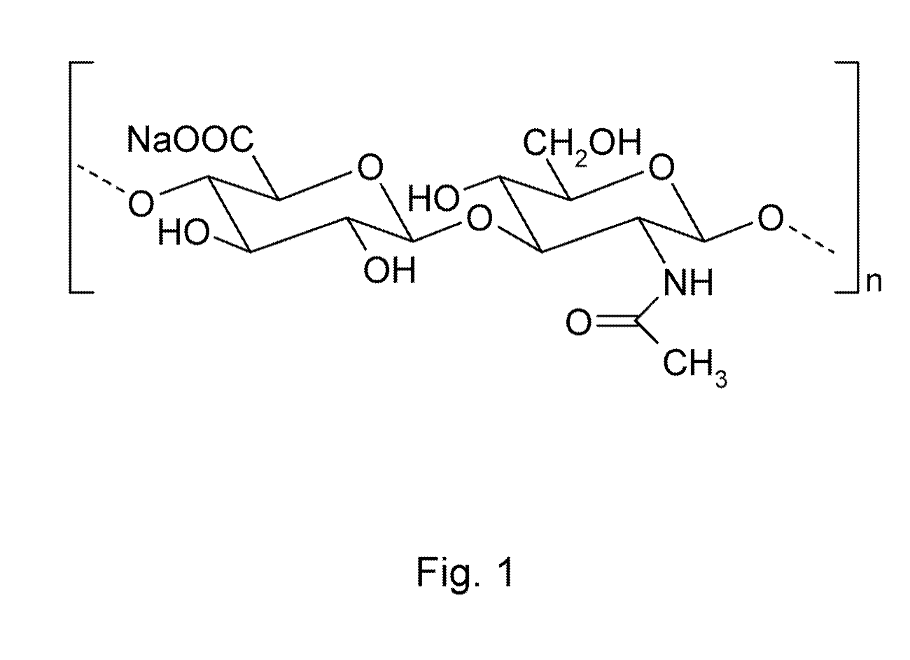

The term “hyaluronic acid” is defined herein as an unsulphated glycosaminoglycan composed of repeating disaccharide units of N-acetylglucosamine (GlcNAc) and glucuronic acid (GlcUA) linked together by alternating beta-1,4-glycosidic bonds and beta-1,3-glycosidic bonds. Hyaluronic acid is also known as hyaluronan, hyaluronate, or HA. The structural formula of the repeating disaccharide unit of N-acetylglucosamine (GlcNAc) and glucuronic acid (GlcUA) is shown in FIG. 1.

It is understood herein that the term “hyaluronic acid” encompasses a group of unsulphated glycosaminoglycans with different molecular weights or even the degraded fractions of the same. For example, the molecular weight of hyaluronic acid can vary from 800 to 10,000,000 Da, or higher in molecular weight.

Any available hyaluronic acid or salt thereof can be used in the methods of the present invention. Possible sources include connective tissue of vertebrates, human umbilical cord, rooster combs, microorganisms (e.g., Streptococcus), and recombinant microorganisms (e.g., Bacillus). Salts include sodium hyaluronate, potassium hyaluronate, ammonium hyaluronate, calcium hyaluronate, magnesium hyaluronate, zinc hyaluronate, or cobalt hyaluronate.

In a preferred aspect, the hyaluronic acid is obtained naturally or recombinantly from a microbial cell comprising the genetic machinery to produce hyaluronic acid. In a more preferred aspect, the hyaluronic acid is obtained from a Streptococcus cell. In another more preferred aspect, the hyaluronic acid is obtained recombinantly from a Bacillus host cell. In a most preferred aspect, the hyaluronic acid is obtained from a Streptococcus zooepidemicus cell (U.S. Pat. No. 4,801,539, European Patent No, 0694616). In another most preferred aspect, the hyaluronic acid is obtained recombinantly from a Bacillus subtilis or Bacillus licheniformis host cell (WO 03/0175902).

In the methods of the present invention, the average molecular weight of a hyaluronic acid derivative will depend on the average molecular weight of the starting hyaluronic acid. The starting hyaluronic acid can be of one average molecular weight, two or more average molecular weights, or a range of average molecular weights. The choice of the molecular weight of the starting hyaluronic acid will depend on whether the molecular weight of a hyaluronic acid is being increased by elongation using a diamine or whether a branched hyaluronic acid is being made using a polyamine. For the former, a starting hyaluronic acid of 1-2 MDa is preferable. For the latter, the molecular weight can be any molecular weight. The choice of the molecular weight of the starting hyaluronic acid will also depend on the application intended in order to generate desirable viscoelastic, mechanical, stability, and/or matrix/carrier properties.

The average molecular weight of a hyaluronic acid or derivative thereof can be determined using standard methods in the art, such as those described by Ueno et al., 1988, Chem. Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif. Size exclusion chromatography coupled to multi-angle laser light scattering (SEC-MALLS) is a preferred method in the art because it reportedly can measure the molecular weight of hyaluronic acid up to 4 MDa. However, SEC-MALLS can be limited in its use to measure high molecular weights because either the available aqueous SEC column has limited pore size or hyaluronic acid molecules can interwine intra- and inter-molecularly, leading to local heterogeneity and rendering a hyaluronic acid solution liquid non-Newtonian. Nonideal (Newtonian) hyaluronic acid solutions can have difficulty passing through various capillary/interstitial pathways in SEC-MALLS systems, and the pressure/shear of the system may degrade hyaluronic acid (Soltés at 2002, Biomedical Chromatography 16: 459-462; Armstrong et al., 1997, Appl. Environ. Microbiol. 63: 2759-2764). Alternatively, viscosity and sedimentation/centrifugation methods can be used to estimate the molecular weight. See, for example, Hokputsa et al., 2003, Eur. Biophys. J. 32: 450-456 and Soltés et al., 2002, supra.

In the methods of the present invention, the average molecular weight of a starting hyaluronic acid can be in the range of about 800 to about 10,000,000 Da or higher in molecular weight. In a preferred aspect, the average molecular weight of a starting hyaluronic acid is in the range of about 1,000 to about 10,000,000 Da. In a preferred aspect, the average molecular weight of a starting hyaluronic acid is in the range of about 1,000 to about 7,500,000 Da. In another preferred aspect, the average molecular weight of a starting hyaluronic acid is in the range of about 2,000 to about 5,000,000 Da. In another preferred aspect, the average molecular weight of a starting hyaluronic acid is in the range of about 2,000 to about 4,000,000 Da. In another preferred aspect, the average molecular weight of a starting hyaluronic acid is in the range of about 2,000 to about 3,000,000 Da. In another preferred aspect, the average molecular weight of a starting hyaluronic acid is in the range of about 4,000 to about 3,000,000 Da. In another preferred aspect, the average molecular weight of a starting hyaluronic acid is in the range of about 8,000 to about 3,000,000 Da. In another preferred aspect, the average molecular weight of a starting hyaluronic acid is in the range of about 10,000 to about 2,500,000 Da. In another preferred aspect, the average molecular weight of a starting hyaluronic acid is in the range of about 25,000 to about 2,500,000 Da. In another preferred aspect, the average molecular weight of a starting hyaluronic acid is in the range of about 50,000 to about 2,500,000 Da. In another preferred aspect, the average molecular weight of a starting hyaluronic acid is in the range of about 50,000 to about 2,000,000 Da. In another preferred aspect, the average molecular weight of a starting hyaluronic acid is in the range of about 50,000 to about 1,500,000 Da. In another preferred aspect, the average molecular weight of a starting hyaluronic acid is in the range of about 50,000 to about 1,000,000 Da. In another preferred aspect, the average molecular weight of a starting hyaluronic acid is in the range of about 50,000 to about 500,000 Da.

The level of hyaluronic acid may be determined according to the modified carbazole method (Bitter and Muir, 1962, Anal Biochem. 4: 330-334).

The term “diamine” is defined herein as an organic compound composed of two amino groups. In the methods of the present invention, the diamine can be any diamine composed of primary amines, secondary amines, or a combination of a primary amine(s) and a secondary amine(s).

In a preferred aspect, the amino groups of the diamine are primary amino groups. In another preferred aspect, the diamine is selected from the group consisting of an aliphatic diamine, aromatic diamine, and heteroatomic diamine.

For example, the aliphatic diamine can be 1,3-diaminopropane, 1,4-diaminobutane, 1,5-diaminopentane, 1,6-diaminohexane, 1,7-diaminoheptane, 1,8-diaminooctane, or lysyl-glycyl-lysine tripeptide; the aromatic diamine can be 1,4-diaminobenzene, 1,4-diaminomethylbenzene, or their branched, cyclized, substituted, oxidized, or dehydrogenated derivatives or analogs; and the heteroatomic diamine can be 2,5-diaminofuran, 2,5-diaminodioxin, or a glucosamine dimer. However, any diamine can be used in practicing the methods of the present invention.

In a more preferred aspect, the diamine is selected from the group consisting of 1,3-diaminopropane, 1,4-butane, 1,5-diaminopentane, 1,6-diaminohexane, 1,7-diaminoheptane, and 1,8-diaminooctane.

The term “polyamine” is defined herein as an organic compound composed of three or more amino groups. In the methods of the present invention, the polyamine can be any polyamine composed of primary amines, secondary amines, or a combination of one or more primary amines and secondary amines.

In a preferred aspect, the amino groups of the polyamine are primary amino groups. In another preferred aspect, the polyamine is selected from the group consisting of an aliphatic polyamine, aromatic polyamine, and heteroatomic polyamine.

For example, the aliphatic polyamine can be 1,3-diamino-2-aminomethyl-propane, 1,7-diamino-4-aminomethyl-heptane, 1,10-diamino-4,7-diaminomethyl-decane, other triamino-n-alkane, tetraminoalkane, triamino-alkene, tetraminoalkyne, or their branched, cyclized, substituted, oxidized, or dehydrogenated derivatives or analogs; the aromatic polymine can be 1,3,5-triaminobenzene, 1,2,4,5-tetraminobenzene, 1,3,5-triaminomethylbenzene, 1,2,4,5-tetraminomethylbenzene, or their branched, cyclized, substituted, oxidized, or dehydrogenated derivatives or analogs; and the heteroatomic polyamine can be 2,3,4,5-tetraminofuran, 2,3,5,6-tetraminodioxin, chitosan, polylysine, or lysine-containing polypeptides. However, any polyamine can be used in practicing the methods of the present invention.

In a more preferred aspect, the polyamine is poly-L-lysine or a polylysine-containing polypeptide.

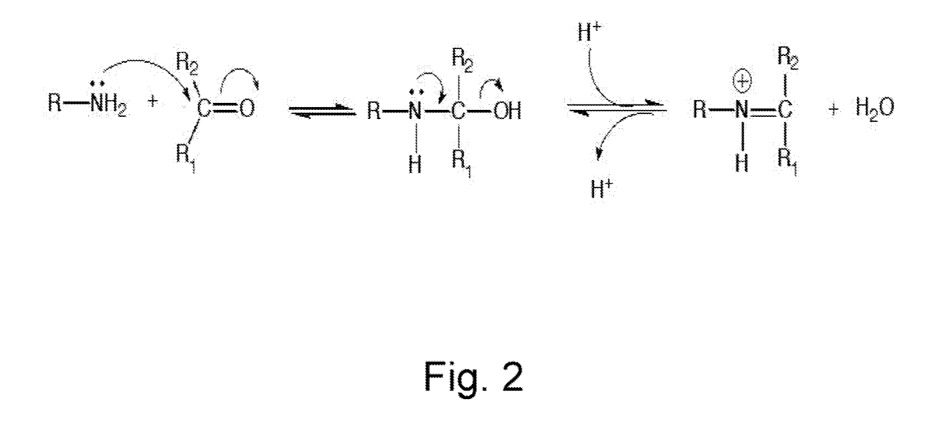

In the methods of the present invention, a hyaluronic acid is reacted with a diamine, a polyamine, or a combination thereof according to the reaction shown in FIG. 2 to produce an imine. The reducing group, e.g., aldehyde or C₁OH in the cyclized hemiacetal form, may be either from N-acetylglucosamine or glucuronic acid depending on which group is at the terminus of hyaluronic acid. The optimal pH for producing an imine is preferably in the slightly acidic pH range, e.g., pH at about 4-6.

Combinations of diamines, polyamines, a diamine and a polyamine, or diamines and polyamines can be used in the methods of the present invention. In a preferred aspect, the reaction is composed of one diamine or one polyamine.

In the methods of the present invention, the concentration of a hyaluronic acid is preferably in the range of about 1 nM to about 10 mM. The choice of concentration will depend on the molecular weight of the hyaluronic acid. For example, a hyaluronic acid with a molecular weight of 1 MDa will likely require a lower concentration, e.g., 1 μM, compared to a hyaluronic acid with a molecular weight of 1000 Da. However, any concentration of a hyaluronic acid may be used in the methods of present invention as long as the dissolved hyaluronic acid has a reasonable viscosity. The concentration of a diamine and/or a polyamine will be in molar excess as described below.

In order to optimize conversion of a starting hyaluronic acid to a derivative of a diamine, a polyamine, or a combination thereof, the molar concentration of the starting hyaluronic acid must be in sufficient excess relative to the molar concentration of the amino groups of the diamine, the polyamine, or the combination thereof to minimize the amount of unreacted hyaluronic acid at the end of the reaction.

For a diamine, the molar ratio of a hyaluronic acid to a diamine is preferably at least about 4:1, more preferably at least about 3.5:1, even more preferably at least about 3:1, and most preferably at least about 2.5:1.

For a polyamine, the molar ratio of a hyaluronic acid to a polyamine will depend on the desired degree of derivatization of the polyamine with the hyaluronic acid. For example, where the desired degree of derivatization of a polyamine with a hyaluronic acid is two hyaluronic acid molecules per molecule of polyamine, the ratio of the hyaluronic acid to the polyamine on a molar basis is preferably at least about 4:1, more preferably at least about 3.5:1, even more preferably at least about 3:1, and most preferably at least about 2.5:1. For higher degrees of derivatization or total derivatization of every amine of the polyamine, the molar ratio would need to be adjusted accordingly to higher molar ratios.

For a combination of a diamine and a polyamine, the molar ratio of a hyaluronic acid to the diamine and the polyamine will again depend on the desired degree of derivatization of the polyamine with the hyaluronic acid. For example, where the desired degree of derivatization of a diamine with hyaluronic acid is two hyaluronic acid molecules per molecule of diamine and the desired degree of derivatization of a polyamine with hyaluronic acid is two hyaluronic acid molecules per molecule of polyamine, the ratio of the hyaluronic acid to the combination of diamine and polyamine on a molar basis (assuming equal concentrations of the diamine and the polyamine) is preferably at least about 8:1, more preferably at least about 7:1, even more preferably at least about 6:1, and most preferably at least about 5:1. Again, for higher degrees of derivatization or total derivatization of every amine of the polyamine, the molar ratios would need to be adjusted accordingly to higher molar ratios. In addition, depending on the molar ratio of the diamine and the polyamine, the molar ratio will need further consideration.

In a preferred aspect, the molar ratio of a diamine to a polyamine is preferably about 1:1000, more preferably about 1:500, more preferably about 1:250, more preferably about 1:100, more preferably about 1:50, more preferably about 1:25, more preferably about 1:10, even more preferably about 1:5, most preferably about 1:2.5, and even most preferably 1:1. In another preferred aspect, the molar ratio of a polyamine to a diamine is preferably about 1:1000, more preferably about 1:500, more preferably about 1:250, more preferably about 1:100, more preferably about 1:50, more preferably about 1:25, more preferably about 1:10, even more preferably about 1:5, most preferably about 1:2.5, and even most preferably 1:1. However, in the methods of the present invention, any desirable molar ratio of a diamine and a polyamine can be used.

It is recognized that depending on the physical properties of the diamine or the polyamine (e.g., water solubility) and whether the amino groups are primary or secondary amino groups, or a combination thereof, the molar ratio of the hyaluronic acid to the diamine, the polyamine, or the combination thereof, may need to be adjusted accordingly depending on the accessibility of the reducing group of a hyaluronic acid to an amino group.

In practicing the methods of the present invention with a polyamine, it may be desirable to multiply the molecular weight of the starting hyaluronic acid, e.g., triple, quadruple, etc. In such a situation, higher ratios of a hyaluronic acid to a polyamine will be required. The optimum ratio can be determined empirically by those skilled in the art.

The reaction is generally conducted in a liquid solution composed of water. The aqueous solution may be supplemented with an organic solvent to increase the solubility of the diamine, the polyamine, or the combination thereof. For example, organic solvents such as an alcohol (e.g., methanol, ethanol, propanol, and others alcohols), ketone (e.g., acetone), and other common organic solvents can be used. Alternatively, the liquid solution can be primarily an organic solvent such as dioxin, furan, dimethylformamide (DMF), and dimethylsulfoxide (DMSO). The organic solvent may be supplemented with water.

In the methods of the present invention, the liquid solution of step (a) is preferably prepared by dissolving a hyaluronic acid in water, e.g., deionized water, to form an aqueous liquid comprising hyaluronic acid. The water is either buffered or sodium hydroxide is added to the aqueous liquid comprising hyaluronic add, so that the hydroxide groups of the hyaluronic acid are deprotonated. The aqueous liquid is left for a period of time at a low temperature to insure uniform solvation of the hyaluronic acid. Then a diamine, a polyamine, or a combination thereof is added. After complete addition of the diamine, the polyamine, or the combination thereof, the liquid reaction mixture is stirred or shaken for sufficient time to insure conversion to an imine. The time for the reaction can be a few minutes up to a few hours depending on the concentration of the reactants, temperature and pH.

The pH of the reaction of a hyaluronic acid with a diamine, a polyamine, or a combination thereof is maintained preferably between about 4 and about 9, more preferably between about 4 and about 8, even more preferably between about 4 and about 7, and most preferably between about 5 and about 6. The pH can be maintained either by buffer and/or by addition of dilute acid (e.g., HCl) or base (e.g., sodium hydroxide).

The temperature of the reaction of a hyaluronic acid with a diamine, a polyamine, or a combination thereof is maintained preferably between about 0° C. and about 100° C., more preferably between about 10° C. and about 80° C., even more preferably between about 15° C. and about 60° C., most preferably between about 20° C. and about 50° C., and even most preferably between about 25° C. and about 40° C.

The term “imine” or “Schiff base” is defined herein as a functional group or type of chemical compound containing a carbon-nitrogen double bond with the nitrogen atom of an amine connected to an aryl group or an alkyl group but not hydrogen, as shown below.

R₁R₂C═N—R₃

wherein R₁, R₂, and R₃ are selected from the group consisting of hydrogen, carbon-anchored groups (alkyl, benzyl, carbonyl, cyanide, carboxyl, and substituted derivatives/analogs), oxygen-anchored groups (hydroxyl, ether, ester, and substituted derivatives/analogs), nitrogen-anchored groups (amine, amide, and substituted derivatives/analogs), and other atom-anchored groups (halide, sulfonyl, sulfate, phosphate, and substituted derivatives/analogs). Imines can be synthesized from an aromatic amine and a carbonyl compound in a nucleophilic addition to a hemiaminal followed elimination of water to the imine. The Schiff base is synonymous with an azomethine.

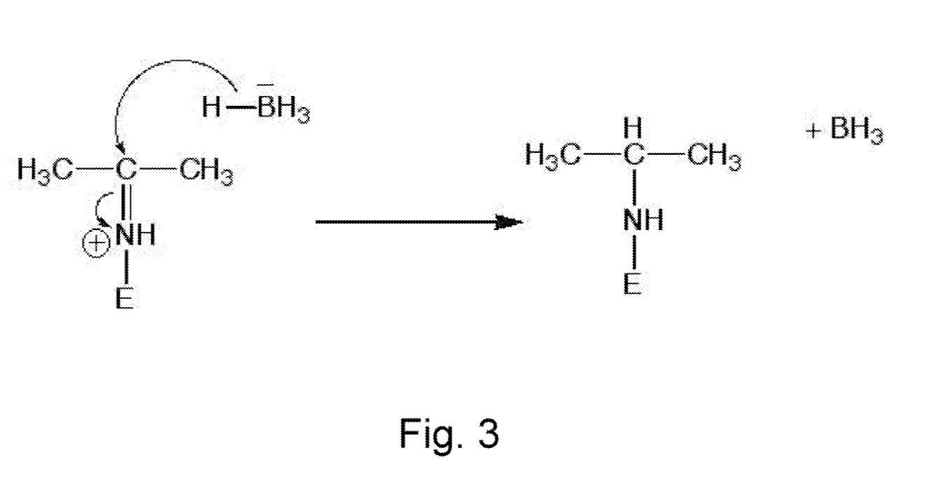

In the methods of the present invention, the reduction of step (b) is performed to reduce or hydrogenate the C═N double bond to a C—N single bond. This is accomplished using a reductant/electron-donor/hydrogenating agent (hereinafter “reductant”). The reduction is preferably conducted in an aqueous solution at a pH and temperature suitable for the reduction. The aqueous solution is preferably either buffered or a dilute acid (e.g., HCl) or base (e.g., sodium hydroxide) is added to maintain the pH. After complete addition of the reductant the liquid reaction mixture is stirred or shaken to insure maximal conversion of the C═N double bond to a C—N single bond. The time for the reduction can be a few minutes up to a few hours depending on the concentrations of the imine and reductant, temperature, and pH. An example of a reduction using borohydride as the reductant is shown in FIG. 3.

In the methods of the present invention, the reduction can be performed by any method known in the art. In a preferred aspect, the reduction is performed with a chemical reductant. In another preferred aspect, the reduction is performed by electrochemical reduction.

When the reduction is performed with a chemical reductant, any suitable chemical reductant known in the art can be used that reduces an imine to an amine. The chemical reductant can be selected from the group consisting of a hydride, metal hydride, metal/hydrogen, and sulfhydryl-like reductant. In a preferred aspect, the chemical reductant is selected from the group consisting of sodium cyanoborohydride (NaCNBH₂), sodium borohydride (NaBH₄), lithium aluminum hydride (LiAlH₄), hydroxycyclopentadienyl ruthenium hydride, Raney nickel and H₂, and sodium dithionite. See, for example, Casey et al., 2006, J. Am. Chem. Soc. 128: 2286-2293, Abdel-Magid et al., 1996, J. Org. Chem., 61: 3849-3862, Pojer, 1979, Aust. J. Chem. 32: 201-204.

The reduction can also be performed electrochemically using methods known in the art. See, for example; Boettcher et al., 1997, Inorg. Chem. 36: 2498-2504.

The pH of the reduction reaction will depend on the reductant used. The pH is maintained preferably between about 4 and about 10, more preferably between about 4 and about 9, even more preferably between about 5 and about 9, and most preferably between about 6 and about 8. The pH can be maintained either by buffer and/or by addition of dilute sodium hydroxide.

The temperature of the reduction reaction is maintained preferably between about 0° C. and about 100° C., more preferably between about 10° C. and about 80° C., even more preferably between about 15° C. and about 60° C., most preferably between about 20° C. and about 50° C., and even most preferably between about 25° C. and about 40° C.

The average molecular weight of the hyaluronic acid derivative can then be determined according to the methods described herein.

The average molecular weight of the hyaluronic acid derivative can be in the range of about 800 to about 20,000,000 Da, or higher in molecular weight. In a preferred aspect, the average molecular weight of the hyaluronic acid derivative is in the range of about 1,000 to about 20,000,000 Da. In another preferred aspect, the average molecular weight of the hyaluronic acid derivative is in the range of about 1,000 to about 15,000,000 Da. In another preferred aspect, the average molecular weight of the hyaluronic acid derivative is in the range of about 1,000 to about 10,000,000 Da. In another preferred aspect, the average molecular weight of the hyaluronic acid derivative is in the range of about 2,000 to about 10,000,000 Da. In another preferred aspect, the average molecular weight of the hyaluronic acid derivative is in the range of about 2,000 to about 8,000,000 Da. In another preferred aspect, the average molecular weight of the hyaluronic acid derivative is in the range of about 2,000 to about 6,000,000 Da. In another preferred aspect, the average molecular weight of the hyaluronic acid derivative is in the range of about 4,000 to about 6,000,000 Da. In another preferred aspect, the average molecular weight of the hyaluronic acid derivative is in the range of about 8,000 to about 6,000,000 Da. In another preferred aspect, the average molecular weight of the hyaluronic acid derivative is in the range of about 10,000 to about 5,000,000 Da. In another preferred aspect, the average molecular weight of the hyaluronic acid derivative is in the range of about 25,000 to about 5,000,000 Da. In another preferred aspect, the average molecular weight of the hyaluronic acid derivative is in the range of about 50,000 to about 5,000,000 Da. In another preferred aspect, the average molecular weight of the hyaluronic acid derivative is in the range of about 50,000 to about 4,000,000 Da. In another preferred aspect, the average molecular weight of the hyaluronic acid derivative is in the range of about 50,000 to about 3,000,000 Da. In another preferred aspect, the average molecular weight of the hyaluronic acid derivative is in the range of about 50,000 to about 2,000,000 Da. In another preferred aspect, the average molecular weight of the hyaluronic acid derivative is in the range of about 50,000 to about 1,000,000 Da. In another preferred aspect, the average molecular weight of the hyaluronic acid derivative is in the range of about 50,000 to about 500,000 Da.

The resulting hyaluronic acid derivative may be recovered by methods known in the art. See, for example, U.S. Pat. No. 5,023,175 and Radaeva et al., 1997, Prikl. Biokhim. Mikrobiol. 33: 133-137. For example, the hyaluronic add derivative may be recovered by conventional procedures including, but not limited to, centrifugation, filtration, extraction, spray-drying, evaporation, or precipitation. The isolated hyaluronic acid derivative may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction (see, e.g., Protein Purification, J.-C. Janson and Lars Ryden, editors, VCH Publishers, New York, 1989).

For example, after the reduction is completed, the hyaluronic acid derivative can be precipitated by addition of an excess of an organic solvent like ethanol, acetone, methanol, or isopropyl alcohol. For purification of the derivatized product, it can be centrifuged and washed with a solvent such as ethanol, methanol, or acetone. The product may then be dialyzed to provide a substantially pure hyaluronic acid derivative.

The hyaluronic derivatives can be characterized by proton or carbon-13 NMR by determining specific chemical shifts corresponding to the aminated sorbitol (glucitol), which are different from those of the pyranosyl beta-1,3-N-acetyl glucosamine or beta-1,4-glucuronic acid unit of hyaluronic acid, or other spectroscopic methods developed for glucose and its derivatives (McNichols and Cote, 2000, Journal of Biomedical Optics 5: 5-16), or by the loss of hyaluronic acid reducing end as detected by reducing sugar-specific reagents such as p-hydroxybenzoic acid hydrazide (Schülein, 1997, J. Biotechnol. 57: 71-81).

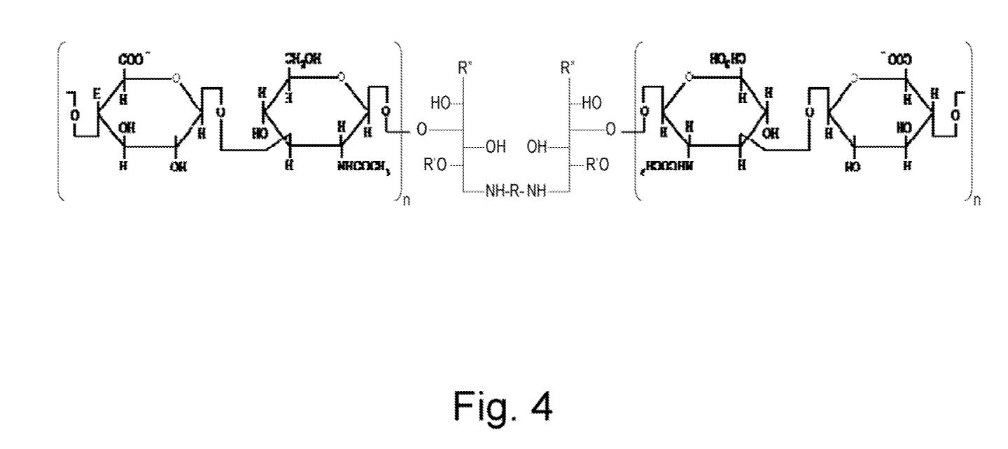

The present invention also relates to isolated derivatives of a hyaluronic acid, comprising the hyaluronic acid and a diamine, a polyamine, or a combination thereof. For example, an isolated hyaluronic acid derivative may have the structure HA-CH₂—NH—R—NH—CH₂—HA for a diamine and HA-CH₂—NH—R(—NH—CH₂—HA)-NH—CH₂—HA for a polyamine, wherein HA is hyaluronic acid and R is the rest of the structure of a diamine or a polyamine. An example of a derivative of hyaluronic acid according to the present invention is shown in FIG. 4. The formation of a Schiff's base of a diamine, for example, with the reducing end (aldose) of a hyaluronic acid leads to the opening of the hyaluronic acid end's pyranose ring, and the Schiff base reduction leads to the formation of the corresponding sorbitol moiety.

Derivatives of a hyaluronic acid and a diamine comprise or consist of two hyaluronic acid molecules per molecule of diamine.

Derivatives of a hyaluronic acid and a polyamine comprise or consist of two or more hyaluronic acid molecules per molecule of polyamine. In a preferred aspect, a derivative of a hyaluronic acid and a polyamine comprises or consists of at least two hyaluronic acid molecules per molecule of polyamine. In another preferred aspect, a derivative of a hyaluronic acid and a polyamine comprises or consists of at least three hyaluronic acid molecules per molecule of polyamine. In another preferred aspect, a derivative of a hyaluronic acid and a polyamine comprises or consists of at least four hyaluronic acid molecules per molecule of polyamine. In another preferred aspect, a derivative of a hyaluronic acid and a polyamine comprises or consists of at least five hyaluronic acid molecules per molecule of polyamine. In another preferred aspect, a derivative of a hyaluronic acid and a polyamine comprises or consists of at least six hyaluronic acid molecules per molecule of polyamine. In another preferred aspect, a derivative of a hyaluronic acid and a polyamine comprises or consists of at least seven hyaluronic acid molecules per molecule of polyamine. In another preferred aspect, a derivative of a hyaluronic acid and a polyamine comprises or consists of at least eight hyaluronic acid molecules per molecule of polyamine. In another preferred aspect, a derivative of a hyaluronic acid and a polyamine comprises or consists of at least nine hyaluronic acid molecules per molecule of polyamine. In another preferred aspect, a derivative of a hyaluronic acid and a polyamine comprises or consists of at least ten hyaluronic acid molecules per molecule of polyamine.

In another preferred aspect, a derivative of a hyaluronic acid and a polyamine comprises or consists of two hyaluronic acid molecules per molecule of polyamine. In another preferred aspect, a derivative of a hyaluronic acid and a polyamine comprises or consists of three hyaluronic acid molecules per molecule of polyamine. In another preferred aspect, a derivative of a hyaluronic acid and a polyamine comprises or consists of four hyaluronic acid molecules per molecule of polyamine. In another preferred aspect, a derivative of a hyaluronic acid and a polyamine comprises or consists of five hyaluronic acid molecules per molecule of polyamine. In another preferred aspect, a derivative of a hyaluronic acid and a polyamine comprises or consists of six hyaluronic acid molecules per molecule of polyamine. In another preferred aspect, a derivative of a hyaluronic acid and a polyamine comprises or consists of seven hyaluronic acid molecules per molecule of polyamine. In another preferred aspect, a derivative of a hyaluronic acid and a polyamine comprises or consists of eight hyaluronic acid molecules per molecule of polyamine. In another preferred aspect, a derivative of a hyaluronic acid and a polyamine comprises or consists of nine hyaluronic acid molecules per molecule of polyamine. In another preferred aspect, a derivative of a hyaluronic acid and a polyamine comprises or consists of ten hyaluronic acid molecules per molecule of polyamine.

Derivatives of a hyaluronic acid and a combination of a diamine and a polyamine comprise or consist of two hyaluronic add molecules per molecule of diamine and two or more hyaluronic acid molecules per molecule of polyamine. In a preferred aspect, a derivative of a hyaluronic acid and a combination of a diamine and a polyamine comprises or consists of two hyaluronic acid molecules per molecule of diamine and at least two hyaluronic acid molecules per molecule of polyamine. In another preferred aspect, a derivative of a hyaluronic acid and a combination of a diamine and a polyamine comprises or consists of two hyaluronic acid molecules per molecule of diamine and at least three hyaluronic acid molecules per molecule of polyamine. In another preferred aspect, a derivative of a hyaluronic add and a combination of a diamine and a polyamine comprises or consists of two hyaluronic acid molecules per molecule of diamine and at least four hyaluronic acid molecules per molecule of polyamine. In another preferred aspect, a derivative of a hyaluronic acid and a combination of a diamine and a polyamine comprises or consists of two hyaluronic acid molecules per molecule of diamine and at least five hyaluronic acid molecules per molecule of polyamine. In another preferred aspect, a derivative of a hyaluronic acid and a combination of a diamine and a polyamine comprises or consists of two hyaluronic acid molecules per molecule of diamine and at least six hyaluronic acid molecules per molecule of polyamine. In another preferred aspect, a derivative of a hyaluronic acid and a combination of a diamine and a polyamine comprises or consists of two hyaluronic acid molecules per molecule of diamine and at least seven hyaluronic acid molecules per molecule of polyamine. In another preferred aspect, a derivative of a hyaluronic acid and a combination of a diamine and a polyamine comprises or consists of two hyaluronic acid molecules per molecule of diamine and at least eight hyaluronic acid molecules per molecule of polyamine. In another preferred aspect, a derivative of a hyaluronic acid and a combination of a diamine and a polyamine comprises or consists of two hyaluronic acid molecules per molecule of diamine and at least nine hyaluronic acid molecules per molecule of polyamine. In another preferred aspect, a derivative of a hyaluronic acid and a combination of a diamine and a polyamine comprises or consists of two hyaluronic acid molecules per molecule of diamine and at least ten hyaluronic acid molecules per molecule of polyamine.

In another preferred aspect, a derivative of a hyaluronic acid and a combination of a diamine and a polyamine comprises or consists of two hyaluronic acid molecules per molecule of diamine and two hyaluronic acid molecules per molecule of polyamine. In another preferred aspect, a derivative of a hyaluronic acid and a combination of a diamine and a polyamine comprises or consists of two hyaluronic acid molecules per molecule of diamine and three hyaluronic acid molecules per molecule of polyamine. In another preferred aspect, a derivative of a hyaluronic acid and a combination of a diamine and a polyamine comprises or consists of two hyaluronic acid molecules per molecule of diamine and four hyaluronic acid molecules per molecule of polyamine. In another preferred aspect, a derivative of a hyaluronic add and a combination of a diamine and a polyamine comprises or consists of two hyaluronic acid molecules per molecule of diamine and five hyaluronic acid molecules per molecule of polyamine. In another preferred aspect, a derivative of a hyaluronic acid and a combination of a diamine and a polyamine comprises or consists of two hyaluronic acid molecules per molecule of diamine and six hyaluronic acid molecules per molecule of polyamine. In another preferred aspect, a derivative of a hyaluronic acid and a combination of a diamine and a polyamine comprises or consists of two hyaluronic acid molecules per molecule of diamine and seven hyaluronic acid molecules per molecule of polyamine. In another preferred aspect, a derivative of a hyaluronic acid and a combination of a diamine and a polyamine comprises or consists of two hyaluronic acid molecules per molecule of diamine and eight hyaluronic acid molecules per molecule of polyamine. In another preferred aspect, a derivative of a hyaluronic acid and a combination of a diamine and a polyamine comprises or consists of two hyaluronic acid molecules per molecule of diamine and nine hyaluronic acid molecules per molecule of polyamine. In another preferred aspect, a derivative of a hyaluronic acid and a combination of a diamine and a polyamine comprises or consists of two hyaluronic acid molecules per molecule of diamine and ten hyaluronic acid molecules per molecule of polyamine.

The hyaluronic acid derivatives of the present invention possess several improved properties not associated with natural hyaluronic acid. These improved properties include viscoelastic, mechanical, stability, and/or matrix/carrier properties.

The methods of the present invention can be used to convert a Bacillus-produced hyaluronic acid of 0.7-2 MDa into a hyaluronic acid product of 1.4-4 MDa, which is more desirable for various applications. See, for example, Wobig et al., 1999, Clin Ther. 21: 1549-1562, Armstrong et al., 1997, Applied and Environmental Microbiology 63: 2759-2764; Goa and Benfield, 1994, Drugs 47: 536-566; Swann and Kuo, 1991, Hyaluronic acid, p. 286-305, In D. Byrom (ed.), Biomaterials—novel materials from biological sources, Stockton Press, New York, N.Y. The methods of the present invention can also be used to tailor a hyaluronic acid to a specific molecular weight. For example, with a diamine, an elongated hyaluronic acid can double the molecular weight of the starting material, while with a polyamine, e.g., a triamine, a hyaluronic acid derivative can triple the molecular weight of the starting material.

A hyaluronic acid derivative of the present invention can be in the form of a salt such sodium, potassium, ammonium, calcium, magnesium, zinc, or cobalt.

A hyaluronic acid derivative of the present invention or salt thereof can be crosslinked using reagents and methods known in the art. For example, crosslinking can be prepared with a polyfunctional epoxy compound as disclosed in EP 0 161 887 81. Total or partial crosslinked esters can be prepared with an aliphatic alcohol, and salts of such partial esters with inorganic or organic bases, are disclosed in U.S. Pat. No. 4,957,744. Other ways of cross-linking are disclosed in U.S. Pat. Nos. 5,616,568, 5,652,347, and 5,874,417.

In a preferred aspect, a hyaluronic acid derivative of the present invention or salt thereof is preferably crosslinked with boric acid. In another preferred aspect, a crosslinked hyaluronic acid derivative comprises borate esters.

### Compositions

The present invention also relates to compositions comprising a hyaluronic acid derivative of the present invention.

The compositions comprising a hyaluronic acid derivative may further comprise an inactive component(s), an active component(s), or a combination of an inactive component(s) and an active component(s). The hyaluronic acid derivative may be used as a carrier for the active component(s).

The active component is preferably a pharmacologically active agent. Non-limiting examples of a pharmacologically active agent which may be used in the present invention include, but is not limited to, a protein and/or a peptide drug, such as, human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or derivative thereof, insulin or derivative thereof, atriopeptin-III, monoclonal antibody, tumor necrosis factor, macrophage activating factor, interleukin, tumor degenerating factor, insulin-like growth factor, epidermal growth factor, tissue plasminogen activator, Factor VII, Factor VIII, and urokinase.

The inactive component is preferably a pharmaceutically acceptable carrier. Any pharmaceutically acceptable carrier known in the art may be used.

The compositions of the present invention may further comprise a water-soluble excipient. A water-soluble excipient may be included for the purpose of stabilizing the active ingredients). The excipient may include a protein, e.g., albumin or gelatin; an amino acid, e.g., glycine, alanine, glutamic acid, arginine, or lysine, or a salt thereof; carbohydrate, e.g., glucose, lactose, xylose, galactose, fructose, maltose, saccharose, dextran, mannitol, sorbitol, trehalose, or chondroitin sulphate; an inorganic salt, e.g., phosphate; a surfactant, e.g., TWEEN® (ICI), polyethylene glycol, or a mixture thereof. The excipient or stabilizer may be used in an amount ranging from 0.001 to 99% by weight of the product.

In a preferred aspect, a composition of the present invention comprises a hyaluronic acid derivative and an active component.

In another preferred aspect, a composition of the present invention comprises a hyaluronic acid derivative and an inactive component.

In another preferred aspect, a composition of the present invention comprises a hyaluronic acid derivative, an active component, and an inactive component.

In another preferred aspect, a composition of the present invention comprises an effective amount of a hyaluronic acid derivative and a pharmaceutically acceptable carrier, excipient or diluent.

In another preferred aspect, a pharmaceutical composition comprises an effective amount of a hyaluronic acid derivative as a vehicle and a pharmacologically active agent.

In a preferred aspect, the excipient or diluent is a water-soluble excipient. In a more preferred aspect, the excipient or diluent is lactose.

### Articles

The present invention also relates to articles and materials comprising a hyaluronic acid derivative of the present invention or a composition thereof, e.g., a cosmetic article or a sanitary article (e.g., a medical article or a surgical article).

In a preferred aspect, a cosmetic article comprises as an active ingredient an effective amount of a hyaluronic acid derivative of the present invention or a composition thereof.

In another preferred aspect, a sanitary article comprises a hyaluronic acid derivative of the present invention or a composition thereof. In a more preferred aspect, the sanitary article is selected from the group consisting of a diaper, a sanitary towel, a surgical sponge, a wound healing sponge, or a part comprised in a band aid or other wound dressing material.

The present invention also relates to a medicament capsule, comprising a hyaluronic acid derivative of the present invention or a composition thereof. It will be understood that the term “medicament capsule” encompasses a microcapsule, nanocapsule, microsphere, or nanosphere.

### Uses

A hyaluronic acid derivative of the present invention or a salt thereof may be employed in a wide range of current and developing applications within cosmetics, opthalmology, rheumatology, drug and gene delivery, wound healing, and tissue engineering.

A hyaluronic acid derivative of the present invention or a salt thereof can be used, for example, in the treatment of osteoarthritis, cancer, ophtalmic conditions, angiogenesis, hair loss or baldness, wounds, or dry skin.

A hyaluronic acid derivative of the present invention or a salt thereof may also be used, for example, for performing dermal or transdermal administration of a pharmacologically active agent, or dermal administration of a cosmetic.

The present invention is further described by the following examples which should not be construed as limiting the scope of the invention.

### EXAMPLES

Chemicals used as buffers and substrates were commercial products of at least reagent grade.

### Solutions

Mixtures of hyaluronic acid (sodium salt) of 0.22 MDa, 0.59 MDa, and 0.81 MDa (medical grade, LifeCore Biomedical, Inc., Chaska, Minn., USA) were prepared by mixing 20 g, 10 g, and 5 g, respectively, in 1.0 liter of glass-distilled water with reducing end concentrations of approximately 89-91 μM, approximately 17 μM, and approximately 6.2 μM, respectively. The molecular weight provided by the manufacturer was used to calculate the concentration of reducing ends, which are equal to the molarity of the hyaluronic acid, assuming that each hyaluronic acid molecule is linear and had only one reducing end.

Poly-L-lysine (polyK) stock solution (0.5 mM) was made by dissolving 8.8 mg (DP 401-453, MW 84-95 kDa. Sigma Chemical Co., St. Louis, Mo., USA) in 0.2 ml of glass-distilled water.

Buffer stock solution was made by mixing 41.6 μl of 10×PBS (phosphate buffered saline composed per liter of 80 g of NaCl, 2.0 g of KCl, 14.4 g of Na₂HPO₄, and 2.4 g of KH₂PO₄), 4.8 μl of 0.1 M sodium borate pH 9.5, 46.4 mg of sodium chloride, and 124.8 μl of glass-distilled water.

Sodium cyanoborohydride (NaCNBH₃) stock solution (2 M) was made just before use by dissolving 17.9 mg of sodium cyanoborohydride (95% purity, Aldrich Chemical Co., Inc., Milwaukee, Wis., USA) in 135.5 μl of glass-distilled water.

### Example 1

### Derivatization of Hyaluronic Acid with Polylysine

A mixture of 200 μl of the 0.22 MDa hyaluronic acid, 22.3 μl of buffer stock solution to adjust the pH to approximately 8.5 with a final sodium chloride concentration of approximately 0.5 M, and 2 μl of poly-L-lysine stock solution with a final concentration of approximately 5 μM for poly-L-lysine and approximately 3 mM for lysine unit was incubated in a 1.7-ml microcentrifuge tube at 50° C. with mixing at 130 rpm. After 6 days (with daily pipet mixing), 12 μl of the NaCNBH₃ stock solution was added to a final concentration of approximately 0.1 M, followed by 3 days of incubation at 50° C. with daily pipet mixing. Then approximately 1.5 mg of NaCNBH₃ powder was added, corresponding to approximately 0.1 M fresh NaCNBH₃, followed by 4 days of incubation at 50° C. with daily pipet mixing. Hyaluronic acid of 0.59 and 0.81 MDa were also tested under the same conditions. Solutions in the absence of poly-L-lysine and NaCNBH₃ served as controls.

For small solutions, the capillarity of the solution was used to compare viscosity of the hyaluronic acid reaction products. A Pasteur glass pipet (diameter of approximately 1 mm) was immersed slightly below the surface of solution to suck in liquid. After 2 minutes, the stationary height (h) and the volume of risen liquid were measured in triplicate. Based on the equations below (Pelofsky, 1966, J. Chem. Eng. Data 11: 394-397), a larger viscosity (η) would lead to a lower h:

h=4σ cos β/(γd), wherein σ is surface tension, β is contact angle, d is diameter, and γ is specific weight wherein the logarithm of σ is inversely proportional to viscosity.

For samples of larger volume, viscosity in cP was measured using a Cole Palmer 98936 rotational viscometer (Cole-Parmer Instrument Company, Vernon Hills, Ill., USA) according to the manufacturer's instructions.

Inverting the tubes containing the hyaluronic acid reaction solutions showed that the reactions with poly-L-lysine and NaCNBH₃ appeared more viscous (less fluidic) than the control. Table 1 shows the liquid rising height (h) of the capillarity measurement. A detectable viscosity increase was observed after hyaluronic acid was reacted with poly-L-lysine and NaCNBH₃. The extent of the viscosity increase followed the order of 0.22 MDa HA>0.59 MDa HA>0.81 MDa HA, consistent with the order of the concentration of free reducing ends.

TABLE 1
Capillarity of hyaluronic add solution with or without
polyK/NaCNBH₃ reaction.

| | 0.22 MDa, | | 0.59 MDa, | | 0.81 MDa, | | | |
|---|---|---|---|---|---|---|---|---|
| HA | 20 g/L | | 10 g/L | | 5 g/L | | — | H₂O |
| PolyK/NaCNBH₃ | + | − | + | − | + | − | + | − |
| h, mm | 4.5 | 8.0 | 5.3 | 7.5 | 7.5 | 7.7 | 9.7 | 11 |
| Viscosity, cP | >>145 | 145* | >560 | 560* | ≈250 | 250* | | 1 |

*Measured separately in water by a rotational viscometer.

The observed change in viscosity indicated that the primary amines in poly-L-lysine reacted with the reducing end of hyaluronic acid. The reaction appeared to proceed more readily with hyaluronic acid of a shorter length, which is likely attributable to more available reducing ends for a given hyaluronic acid concentration.

### Example 2

### Derivatization of Hyaluronic Acid with Polylysine and/or 1,8-diaminooctane

In the first experiment, three 0.2 ml solutions were prepared containing 20 WI of 0.22 MDa hyaluronic acid, 0.5 M sodium chloride, 0.3 mM sodium borate, 1% strength of PBS buffer stock solution, with a final pH of 8.6. To the first solution was added 6.6 mM 1,8-diaminooctane (approximately 13 mM —NH₂). To the second solution was added 0.56 μM poly-L-lysine (approximately 0.35 mM —NH₂). To the third solution was added 14 M poly-L-lysine and 6.6 mM 1,8-diaminooctane (approximately 13 mM —NH₂) as well as 0.1 M NaCNBH₃. The solutions were incubated in 1.7-ml microcentrifuge tube at 45° C. for 3 days with daily pipet mixing.

In the second experiment, four 0.2 ml solutions were prepared containing 5 g/l of 0.81 MDa hyaluronic acid, 0.5 M sodium chloride, 0.3 mM sodium borate, ¼ strength of PBS buffer stock solution, with a final pH of 8.6. To the first solution was added 0.31 mM 1,8-diaminooctane (approximately 0.6 mM —NH₂). To the second solution was added 0.1 M NaCNBH₃. To the third solution was added 0.31 mM 1,8-diaminooctane (approximately 0.6 mM —NH₂) and 0.1 M NaCNBH₃. To the fourth solution was added 3.1 μM 1,8-diaminooctane (approximately 6 mM —NH₂) and 0.1 M NaCNBH₃. The solutions were incubated in 1.7-ml microcentrifuge tube at 45° C. for 5 days with daily pipet mixing.

In the first experiment, inverting the reaction tubes showed that the reaction containing poly-L-lysine, 1,8-diaminooctane, and NaCNBH₃ was significantly more viscous than the other two reactions. In the second experiment, inverting the reaction tubes showed that the reaction containing 0.3 mM 1,8-diaminooctane and 0.1 M NaCNBH₃ was more viscous than the other four reactions. The observed change in viscosity indicated that the primary amines in 1,8-diaminooctane reacted with the reducing end of hyaluronic acid.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. A method for preparing a derivative of a hyaluronic acid, comprising:
(a) mixing a liquid solution comprising the hyaluronic acid and a diamine, a polyamine, or a combination thereof at a pH suitable to form an imine;
(b) reducing the imine to an amine with a reductant at a pH suitable to produce the derivative of the hyaluronic acid; and
(c) recovering the derivative of the hyaluronic acid.

2. (canceled)

3. (canceled)

4. (canceled)

5. (canceled)

6. The method of any of claims 1-3, wherein the hyaluronic acid and the diamine in step (a) are present in a molar ratio of the hyaluronic acid to the diamine of at least about 2.5 to 1.

7. The method of any of claims 1, 4, and 5, wherein the hyaluronic acid and the polyamine in step (a) are present in a molar ratio of the hyaluronic acid to the polyamine of at least about 2.5 to 1.

8. The method of any of claims 1-5, wherein the hyaluronic acid and the combination of the diamine and the polyamine in step (a) are present in a molar ratio of the hyaluronic acid to the combination of the diamine and the polyamine of at least about 5 to 1.

9. The method of claim 1, wherein the pH of step (a) is maintained between about 4 to about 9.

10. The method of claim wherein the temperature of step (a) is maintained between about 0° C. to about 100° C.

11. The method of claim 1, wherein the reduction is performed with a chemical reductant or by electrochemical reduction.

12. The method of claim wherein the pH of step (b) is maintained between about 4 to about 10.

13. The method of claim 1, wherein the temperature of step (b) is maintained between about 0° C. to about 100° C.

14. (canceled)

15. The method of claim 1, wherein the derivative of the hyaluronic acid and the diamine comprises or consists of two hyaluronic acid molecules per molecule of diamine.

16. The method of claim wherein the derivative of the hyaluronic acid and the polyamine comprises or consists of at east two hyaluronic acid molecules per molecule of polyamine.

17. The method of claim 1, wherein the derivative of the hyaluronic acid and the combination of the diamine and the polyamine comprises or consists of two hyaluronic acid molecules per molecule of diamine and at least two hyaluronic acid molecules per molecule of polyamine.

18. An isolated derivative of a hyaluronic acid, comprising the hyaluronic acid and a diamine, a polyamine, or a combination thereof.

19. (canceled)

20. (canceled)

21. (canceled)

22. (canceled)

23. The isolated derivative of a hyaluronic acid of claim 18, wherein the derivative of the hyaluronic acid and the diamine comprises or consists of two hyaluronic acid molecules per molecule of diamine.

24. The isolated derivative of a hyaluronic acid of claim 18, wherein the derivative of the hyaluronic acid and the polyamine comprises or consists of at least two hyaluronic acid molecules per molecule of polyamine.

25. The isolated derivative of a hyaluronic acid of claim 18, wherein the derivative of the hyaluronic acid and the combination of the diamine and the polyamine comprises or consists of two hyaluronic acid molecules per molecule of diamine and at least two hyaluronic acid molecules per molecule of polyamine.

26. A composition comprising the hyaluronic acid derivative of claim 18 and an inactive component(s), an active component(s), or a combination of an inactive component(s) and an active component(s)

27. (canceled)

28. (canceled)

29. A cosmetic article comprising a hyaluronic acid derivative of claim 18 or a composition thereof.

30. A sanitary article comprising a hyaluronic acid derivative of claim 18 or a composition thereof.

31. (canceled)

32. A medicament capsule, comprising a hyaluronic acid derivative of claim 18 or a composition thereof.
